(19) United States
(12) Patent Application Publication
(10) Pub. No.: US 2025/0311982 A1
(43) Pub. Date: Oct. 9, 2025

(54) REMOTE MEDICAL SYSTEMS AND METHODS

(71) Applicant: SHANGHAI UNITED IMAGING HEALTHCARE CO., LTD., Shanghai (CN)

(72) Inventors: Chunjiang YAO, Shanghai (CN); Xiaoyang DUAN, Shanghai (CN)

(73) Assignee: SHANGHAI UNITED IMAGING HEALTHCARE CO., LTD., Shanghai (CN)

(21) Appl. No.: 19/241,330

(22) Filed: Jun. 17, 2025

Related U.S. Application Data
(63) Continuation of application No. PCT/CN2024/079729, filed on Mar. 1, 2024.

(30) Foreign Application Priority Data
Mar. 3, 2023 (CN) 202310206442.0

(51) Int. Cl. A61B 5/00; A61B 5/256

(52) U.S. Cl. CPC A61B 5/7435; A61B 5/256; A61B 5/7455; A61B 5/748

(57) ABSTRACT

A method implemented on a first wearable device worn on a first user to be examined may be provided. The method may include receiving a stimulation instruction to apply a target stimulation on the first user from a second wearable device. The stimulation instruction may be invoked by a second user via touching a second position of the second wearable device. The method may also include determining one or more stimulation parameters relating to the target stimulation based on the stimulation instruction. The one or more stimulation parameters may at least include a target body part to be stimulated determined based on the second position. The method may also include performing the target stimulation on the target body part of the first user based on the one or more stimulation parameters. The method may further include determining feedback information of the first user with respect to the target stimulation.

### CROSS-REFERENCE TO RELATED APPLICATIONS

This application is a continuation of International Application No. PCT/CN2024/079729, filed on Mar. 1, 2024, which claims priority of Chinese Patent Application No. 202310206442.0 filed on Mar. 3, 2023, the contents of each of which are incorporated herein by reference.

### TECHNICAL FIELD

The present disclosure relates to medical technology field, and in particular, to remote medical systems and methods.

### BACKGROUND

It is usually time-consuming and inconvenient for patients (especially, the elderly, people with limited mobility, etc.) to go to the hospital for diagnosis and treatment. Recently, remote medical systems have used for providing diagnosis and treatment services for patients.

### SUMMARY

According to an aspect of the present disclosure, a method may be provided. The method may be implemented on a first wearable device worn on a first user to be examined. The method may include receiving, from a second wearable device, a stimulation instruction to apply a target stimulation on the first user. The stimulation instruction may be invoked by a second user via touching a second position of the second wearable device. The method may also include determining one or more stimulation parameters relating to the target stimulation based on the stimulation instruction. The one or more stimulation parameters may at least include a target body part to be stimulated determined based on the second position. The method may also include performing the target stimulation on the target body part of the first user based on the one or more stimulation parameters. The method may further include determining feedback information of the first user with respect to the target stimulation.

In some embodiments, to perform the target stimulation on the target body part of the first user based on the one or more stimulation parameters, the method may include determining a corresponding relationship between positions of the first wearable device and body parts of the first user. The method may also include determining a first position of the first wearable device corresponding to the target body part based on the corresponding relationship and the target body part. The method may further include performing the target stimulation on the target body part of the first user based on the first position and the one or more stimulation parameters.

In some embodiments, to determining a corresponding relationship between positions of the first wearable device and body parts of the first user, the method may include obtaining contour information of the first wearable device. The method may also include determining a surface model relating to the first user based on the contour information. The method may further include determining the first corresponding relationship based on the surface model.

In some embodiments, the stimulation instruction may include touch strength of the touch of the second user on the second position, and the one or more stimulation parameters may further include a stimulation strength of the target stimulation determined based on the touch strength.

In some embodiments, the second wearable device is worn on the second user, the feedback information includes a touch operation input by the first user via touching the first wearable device. The method may include generating a second stimulation instruction to apply a second target stimulation on the second user based on the touch operation. The method may further include transmitting the second stimulation instruction to apply the second target stimulation on the second user to the second wearable device.

In some embodiments, to transmit the second stimulation instruction to apply the second target stimulation on the second user to the second wearable device, the method may include determining whether a second stimulation strength of the second target stimulation is greater than a strength threshold. In response to determining that the second stimulation strength is greater than the strength threshold, the method may include adjusting the second stimulation strength based on an adjustment coefficient and transmitting the second stimulation instruction that includes the adjusted second stimulation strength; or in response to determining that the second stimulation strength is not greater than the strength threshold, the method may include transmitting the second stimulation instruction that includes the second stimulation strength.

In some embodiments, to determine feedback information of the first user with respect to the target stimulation, the method may include obtaining one or more images of the first user captured after the target stimulation is performed on the target body part of the first user. The method may further include determining the feedback information based on the image.

In some embodiments, to determine feedback information of the first user with respect to the target stimulation, the method may include measuring one or more physiological parameters of the first user after the target stimulation is performed on the target body part of the first user using one or more physiological sensors mounted on the first wearable device. The method may further include determining the feedback information based on the one or more physiological parameters.

In some embodiments, the method may further include determining state evaluation information of the first user based on the feedback information, and sending the state evaluation information to a display device of the second user; or the method may further include sending the feedback information to the display device of the second user or the second wearable device.

In some embodiments, the second wearable device may be worn on the second user or a human model.

According to another aspect of the present disclosure, a method may be provided. The method may be implemented on a second wearable device. The method may include receiving a touch operation input by a second user via touching a second position of the second wearable device. The method may include generating a stimulation instruction to apply a target stimulation on a first user based on the touch operation. The method may further include transmitting the stimulation instruction to a first wearable device for directing the first wearable device to apply the target stimulation on the first user.

In some embodiments, the method may further include determining a touch strength of the touch operation of the second user on the second wearable device.

In some embodiments, the stimulation instruction may further comprise a stimulation strength of the target stimulation determined based on the touch strength.

In some embodiments, the method may further include obtaining feedback information of the first user with respect to the target stimulation from the first wearable device. The method may also include determining state evaluation information of the first user based on the feedback information.

In some embodiments, the second wearable device is worn on the second user. The method may further include receiving a second stimulation instruction to apply a second target stimulation on the second user from the first wearable device. The second stimulation instruction may be determined based on feedback information of the first user with respect to the target stimulation. The method may also include performing the second target stimulation on the second user based on the second stimulation instruction.

In some embodiments, the second wearable device may be worn on the second user or a human model.

According to yet another aspect of the present disclosure, a method may be provided. The method may be implemented on a first wearable device worn on a first user to be examined. The method may include receiving a touch operation input by the first user via touching a first position of the first wearable device. The method may also include determining one or more stimulation parameters relating to a target stimulation to be performed on the first user and a second user based on the touch operation. The one or more stimulation parameters may at least include a target body part to be stimulated determined based on the first position. The method may also include performing the target stimulation on the target body part of the first user based on the one or more stimulation parameters. The method may further include transmitting a stimulation instruction to a second wearable device worn on the second user for directing the second wearable device to apply the target stimulation on the target body part of the second user.

In some embodiments, the one or more stimulation parameters further include a stimulation strength of the target stimulation, and the method may include determining whether the stimulation strength of the target stimulation is greater than a strength threshold. In response to determining that the stimulation strength is greater than the strength threshold, the method may include adjusting the stimulation strength based on an adjustment coefficient and transmitting the stimulation instruction that includes the adjusted strength; or in response to determining that the stimulation strength is not greater than the strength threshold, the method may include transmitting the stimulation instruction that includes the stimulation strength.

In some embodiments, the method may include determining feedback information of the first user with respect to the target stimulation.

In some embodiments, to determine feedback information of the first user with respect to the target stimulation, the method may include obtaining one or more images of the first user after the target stimulation is performed on the target body part of the first user. The method may further include determining the feedback information based on the one or more images.

In some embodiments, to determine feedback information of the first user with respect to the target stimulation, the method may include measuring one or more physiological parameters of the first user after the target stimulation is performed on the target body part of the first user using one or more physiological sensors mounted on the first wearable device. The method may further include determining the feedback information based on the one or more physiological parameters.

In some embodiments, the method may further include determining state evaluation information of the target body part of the first user based on the feedback information, and sending the state evaluation information to a display device of the second user; or the method may further include sending the feedback information to the display device of the second user or the second wearable device.

In some embodiments, the method may further include obtaining a second image of the first user. The method may further include annotating a lesion region on the second image of the first user based on the state evaluation information of the target body part.

According to yet another aspect of the present disclosure, a first wearable device worn on a first user to be examined may be provided. The first wearable device may include at least one storage device including a set of instructions and at least one processor in communication with the at least one storage device. When executing the set of instructions, the at least one processor is configured to direct the first wearable device to perform the methods implemented on the first wearable device worn.

According to yet another aspect of the present disclosure, a second wearable device may be provided. The second wearable device may comprise at least one storage device including a set of instructions and at least one processor in communication with the at least one storage device. When executing the set of instructions, the at least one processor is configured to direct the second wearable device to perform the methods implemented on the second wearable device worn.

According to yet another aspect of the present disclosure, a non-transitory computer readable medium may be provided. The non-transitory computer readable medium may comprise at least one set of instructions. When executed by one or more processors of a first wearable device worn on a first user to be examined, the at least one set of instructions causes the first wearable device to perform the methods implemented on the first wearable device worn.

According to yet another aspect of the present disclosure, a non-transitory computer readable medium may be provided. The non-transitory computer readable medium may comprise at least one set of instructions. When executed by one or more processors of a second wearable device, the at least one set of instructions causes the second wearable device to perform the methods implemented on the second wearable device worn.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary remote medical system according to some embodiments of the present disclosure;

FIG. 2 is a schematic diagram illustrating an exemplary wearable device according to some embodiments of the present disclosure;

FIG. 3 is a schematic diagram illustrating an exemplary wearable device according to some embodiments of the present disclosure;

FIG. 4 is a flowchart illustrating an exemplary process for remote diagnosis according to some embodiments of the present disclosure;

FIG. 5 is a schematic diagram illustrating an exemplary process for remote diagnosis according to some embodiments of the present disclosure; and

FIG. 6 is a flowchart illustrating an exemplary process for remote diagnosis according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on wearable devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing wearable device, for execution by the wearable device.

Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or wearable device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, a representation of a subject (e.g., an object, a patient, or a portion thereof) in an image may be referred to as “subject” for brevity. For instance, a representation of an organ, tissue (e.g., a heart, a liver, a lung), or an ROI in an image may be referred to as the organ, tissue, or ROI, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an ROI from the image may be referred to as a segmentation of the ROI for brevity.

In conventional remote medical systems, doctors and patients can only communicate through voice, text, pictures, videos, etc. Therefore, the conventional remote medical systems cannot completely simulate all aspects of outpatient service. For example, the remote medical systems cannot enable the doctors to learn more about the illness of the patients through palpation. Thus, it may be desirable to develop remote medical systems that can provide more comprehensive medical services like actual medical systems, and relevant methods of the remote medical systems.

An aspect of the present disclosure relates to a remote medical system and method. The method may be implemented on a first wearable device and a second wearable device. The first wearable device is worn on a first user (e.g., a patient) to be examined. Specifically, the second wearable device may receive a touch operation input by a second user via touching a position of the second wearable device, and generate a stimulation instruction to apply a target stimulation (e.g., a press) on the first user based on the touch operation. Further, the second wearable device may transmit the stimulation instruction to the first wearable device for directing the first wearable device to apply the target stimulation on the first user. After the first wearable device receives the stimulation instruction, the first wearable device may determine one or more stimulation parameters relating to the target stimulation based on the stimulation instruction. The one or more stimulation parameters may at least include a target body part to be stimulated determined based on the second position. Then, the first wearable device may apply the target stimulation on the target body part of the first user based on the one or more stimulation parameters, and determine feedback information of the first user with respect to the target stimulation.

Compared with the conventional remote medical systems, the remote medical systems and methods of the present disclosure can simulate the palpation process through the first wearable device and the second wearable device, which can improve the diagnosis and treatment accuracy. In addition, in some embodiments, various types of feedback information (e.g., expression information, motion state information, physiological parameter information, voice information, image information, a strength of the discomfort, text information) can be collected by the first wearable device and sent to the second wearable device so that the second user can learn the actual reaction of the first user more accurately.

Another aspect of the present disclosure relates to another remote medical system and method. The method may be implemented on the first wearable device and the second wearable device. The first wearable device may receive a touch operation input by a first user (e.g., a patient) via touching a first position of the first wearable device. The first wearable device may also determine one or more stimulation parameters relating to a target stimulation to be performed on the first user and a second user based on the touch operation. The one or more stimulation parameters may at least include a target body part determined based on the first position. Further, the first wearable device may apply the target stimulation on the target body part of the first user based on the one or more stimulation parameters. The first wearable device may also transmit a stimulation instruction to the second wearable device worn on the second user for directing the second wearable device to apply the target stimulation on the target body part of the second user. In this way, the second user can actually perceive the feeling of the first user, which can improve the diagnosis accuracy.

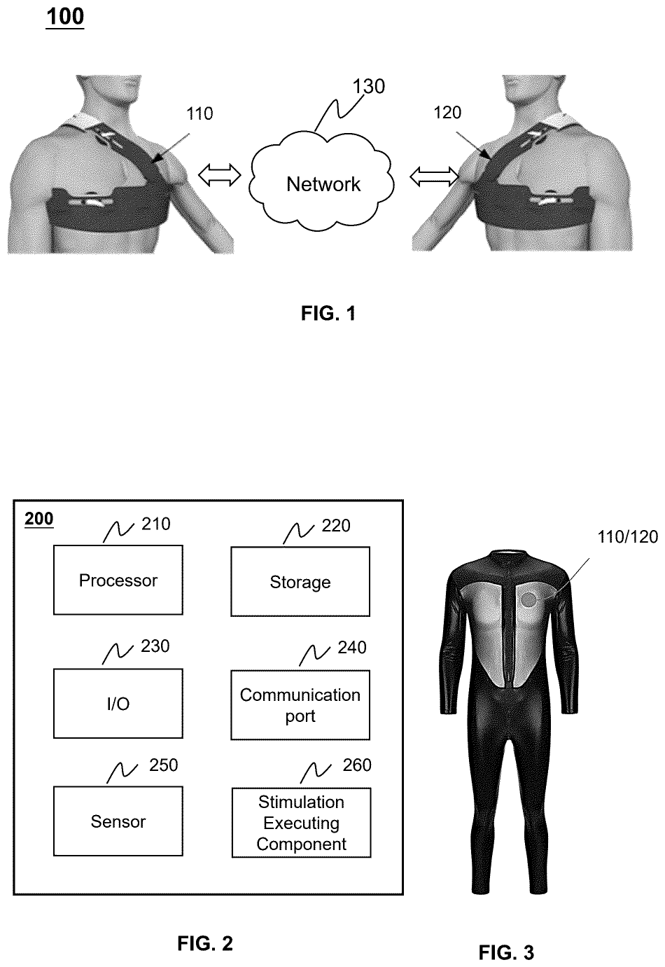

FIG. 1 is a schematic diagram illustrating an exemplary remote medical system 100 according to some embodiments of the present disclosure. As shown in FIG. 1, the remote medical system 100 may include a first wearable device 110, a second wearable device 120, and a network 130. In some embodiments, the first wearable device 110 and the second wearable device 120 may be connected to and/or communicate with each other via the network 130.

The first wearable device 110 may be worn on a first user (e.g., a patient) to be examined. The second wearable device 120 may be worn on a user (e.g., a doctor) or a human model that is near the second user. As used herein, a wearable device may be a portable smart device worn directly on a user or integrated into the user's clothing or accessories. Exemplary wearable devices may include a smart watch, a smart wristband, a smart shoe, a smart sock, a smart glass, a smart helmet, a smart headband, a smart clothing, etc. The types of the first wearable device and the second wearable device may be determined according to specific application environment. For example, in order to collect more user information, each of the first wearable device 110 and the second wearable device 120 may be a tight that covers the whole body as shown in FIG. 3. In some embodiments, the first wearable device 110 and the second wearable device 120 may be of the same type or different types.

In some embodiments, each of the first wearable device 110 and the second wearable device 120 may include one or more components as shown in FIG. 2, for example, a processor, a storage, an I/O, a communication port, a sensor, a stimulation applying component. More descriptions regarding the components of a wearable device may be found elsewhere in the present disclosure. See, e.g., FIG. 2 and relevant descriptions thereof.

The first wearable device 110 and the second wearable device 120 may be configured to assist a user (e.g., a doctor) for performing remote diagnosis. For example, a patient (i.e., the first user) suddenly feels headache and chest tightness, but is unable to go to the hospital. The patient can wear the first wearable device 110 and log into his/her account in a remote medical platform via the first wearable device 110 or another user terminal (e.g., a mobile phone). Then, the patient may point out an uncomfortable body part (i.e., a target body part) (e.g., the heart, the brain) by touching a first position of first wearable device 110 that covers the uncomfortable body part. In some embodiments, the patient may indicate a strength (also referred to as a degree) of the discomfort (e.g., pain) by pressing the first wearable device 110. The first wearable device 110 may receive a touch operation input by the patient via touching the first position of the first wearable device 110, and determine one or more stimulation parameters relating to a target stimulation (e.g., a pressing action) to be performed on the patient based on the touch operation. The one or more stimulation parameters may at least include the target body part of the patient determined based on the first position. Then, the first wearable device 110 may apply the target stimulation on the target body part of the patient based on the one or more stimulation parameters.

In some embodiments, in order to enable the doctor (i.e., the second user) to accurately determine the symptoms and/or the strength of the discomfort of the patient, the first wearable device 110 may transmit a stimulation instruction to the second wearable device 120 worn on the doctor for directing the second wearable device 120 to apply the target stimulation on the target body part of the doctor. Further, the first wearable device 110 may determine feedback information of the patient with respect to the target stimulation, determine state evaluation information of the target body part of the patient based on the feedback information, and send the state evaluation information to a display device of the doctor. The doctor may perform a diagnosis on the patient based on the state evaluation information and/or the feedback information. For example, the doctor determines that patient currently has symptoms of hypertension, gives a prescription for antihypertensive drugs. The patient may go to the nearest pharmacy to buy the drug based on the prescription to relieve headache and chest tightness.

As another example, the doctor needs to review the physical condition of the patient after taking antihypertensive drugs to determine whether it is necessary to reduce the dose of antihypertensive drugs or prescribe another prescription drug for auxiliary treatment. Therefore, the doctor may determine a predict discomfort strength of the patient after the patient takes antihypertensive drugs according to experiences. The doctor may input a touch operation via touching a second position of the second wearable device 120. The second wearable device 120 may generate a stimulation instruction to apply a target stimulation on the patient based on the touch operation. The second wearable device 120 may determine one or more stimulation parameters relating to the target stimulation based on the stimulation instruction, and transmit the stimulation instruction to the first wearable device 110 for directing the first wearable device 110 to apply the target stimulation on the patient. Alternatively or optionally, the second wearable device 120 may send information relating to the touch operation to the first wearable device 110, the first wearable device 110 may determine the one or more stimulation parameters relating to the target stimulation, apply the target stimulation on the patient based on the one or more stimulation parameters.

The one or more stimulation parameters may at least include the target body part to be stimulated determined based on the second position. The first wearable device 110 may apply the target stimulation on the target body part of the patient. The first wearable device 110 may determine feedback information of the patient with respect to the target stimulation. For example, if the feedback information indicates that the actual strength of the discomfort is smaller than the strength of the target stimulation, the doctor may rewrite the prescription to reduce the dose of antihypertensive medication.

In some embodiments, the doctor described in various embodiments of the present disclosure may be a human or an intelligent robot. The intelligent robot may be configured with machine learning models trained using machine learning algorithms and have the same medical capabilities as the doctor.

The network 130 may include any suitable network that can facilitate the exchange of information and/or data for the remote medical system 100. In some embodiments, one or more components of the remote medical system 100 (e.g., the first wearable device 110, the second wearable device 120) may communicate information and/or data with one or more other components of the remote medical system 100 via the network 130.

It should be noted that the above description of the remote medical system 100 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments.

In some embodiments, the remote medical system 100 may include one or more additional components. For example, the remote medical system 100 may further include a first terminal connected with the first wearable device 110 and/or a second terminal connected with the second wearable device 120. In some embodiments, a terminal may include a display device for displaying information (e.g., a video, an image, a text, etc.) relating to the remote medical system 100. Exemplary display devices may include a smartphone, a tablet, a laptop, a desktop computer, a virtual reality device, or the like. In some embodiments, the first terminal may be integrated into the first wearable device 110. In some embodiments, the second terminal may be integrated into the second wearable device 120. For example, the second terminal may be a virtual reality device disposed on the second wearable device 120.

As another example, the remote medical system 100 may further include a storage device for storing data, instructions, and/or any other information.

Additionally or alternatively, one or more components of the remote medical system 100 described above may be omitted. As another example, two or more components of the remote medical system 100 may be integrated into a single component.

FIG. 2 is a schematic diagram illustrating an exemplary wearable device according to some embodiments of the present disclosure. As illustrated in FIG. 2, the wearable device 200 may include a processor 210, a storage 220, an input/output (I/O) 230, a communication port 240, a sensor 250, a stimulation applying component 260.

The processor 210 may execute computer instructions (program code) and perform functions of the wearable device 200 in accordance with techniques described herein. The computer instructions may include routines, programs, objects, components, signals, data structures, procedures, modules, and functions, which perform particular functions described herein. Merely for illustration purposes, only one processor is described in the wearable device 200. However, it should be noted that the wearable device 200 in the present disclosure may also include multiple processors, and thus operations of a method that are performed by one processor as described in the present disclosure may also be jointly or separately performed by the multiple processors.

The storage 220 may store data/information relating to the remote medical system 100. In some embodiments, the storage 220 may include a mass storage device, a removable storage device, a volatile read-and-write memory, a read-only memory (ROM), or the like, or any combination thereof. In some embodiments, the storage 220 may store one or more programs and/or instructions to perform exemplary methods described in the present disclosure.

The I/O 230 may input or output signals, data, or information. In some embodiments, the I/O 230 may enable user interaction with the wearable device 200. In some embodiments, the I/O 230 may include an input device and an output device. The input device may include buttons, touch regions, etc., disposed on the wearable device 200. The output device may include a display, a speaker, or the like.

The communication port 240 may be connected to a network (e.g., the network 130) to facilitate data communications. The communication port 240 may establish connections between the wearable device 200 and other components. The connection may be a wired connection, a wireless connection, or combination of both that enables data transmission and reception.

The sensor 250 may be used to collect information relating to a user who wears the wearable device 200. Merely for illustration purposes, only one sensor is described in the wearable device 200. However, it should be noted that the wearable device 200 in the present disclosure may also include multiple sensors. The sensor 250 may include a capacitance sensor, a pressure sensor, a position sensor, a physiological sensor, or the like, or any combination thereof. The pressure sensor is used to measure a pressure signal exerted by a user on the wearable device 200 and convert the pressure signal into an electrical signal. The pressure sensor may include a pressure sensitive element and a signal processing unit. Exemplary pressure sensors may include a gauge pressure sensor, a differential pressure sensor, an absolute pressure sensor, etc. The capacitance sensor may be a sensor that converts a measured mechanical quantity into a capacitance change. Exemplary capacitance sensors may include a capacitance sensor with variable parameters, a capacitance sensor with a variable electrode spacing. The capacitance sensor with a variable electrode spacing can measure tiny displacements or changes in electrode spacing caused by force, vibration, etc. The physiological sensor may be used for collecting one or more physiological parameters (e.g., a temperature, a blood pressure, a blood oxygen, etc.) of the user. The wearable device 200 may also collect elastic deformation data, hardness data, etc., of body parts of the user. The hardness data of a body part may be determined based on the force acting on the wearable device 200.

The stimulation applying component 260 may be configured to apply stimulation on the user. The type of the stimulation may include a pressing stimulation, an electrical stimulation, a temperature stimulation (which is used to increase or decrease the temperature), a vibration stimulation, medicine application stimulation, or the like. The stimulation applying component 260 may include a pressing component for performing the pressing stimulation, an electrical stimulation component for performing the electrical stimulation, a temperature stimulation component for performing the temperature stimulation, a vibration component for performing the vibration stimulation, a medicine application component for performing the medicine application stimulation, or the like. In some embodiments, one component can function as both the stimulation applying component 260 and the sensor 250. For example, the pressing component can both apply force and measure force, accordingly, the pressing component may be also used as the pressure sensor, that is, the pressing component and the pressure sensor are the same component.

It should be noted that the above description of the wearable device 200 is merely provided for the purposes of illustration, and not intended to limit the scope of the present disclosure. For persons having ordinary skills in the art, multiple variations and modifications may be made under the teachings of the present disclosure.

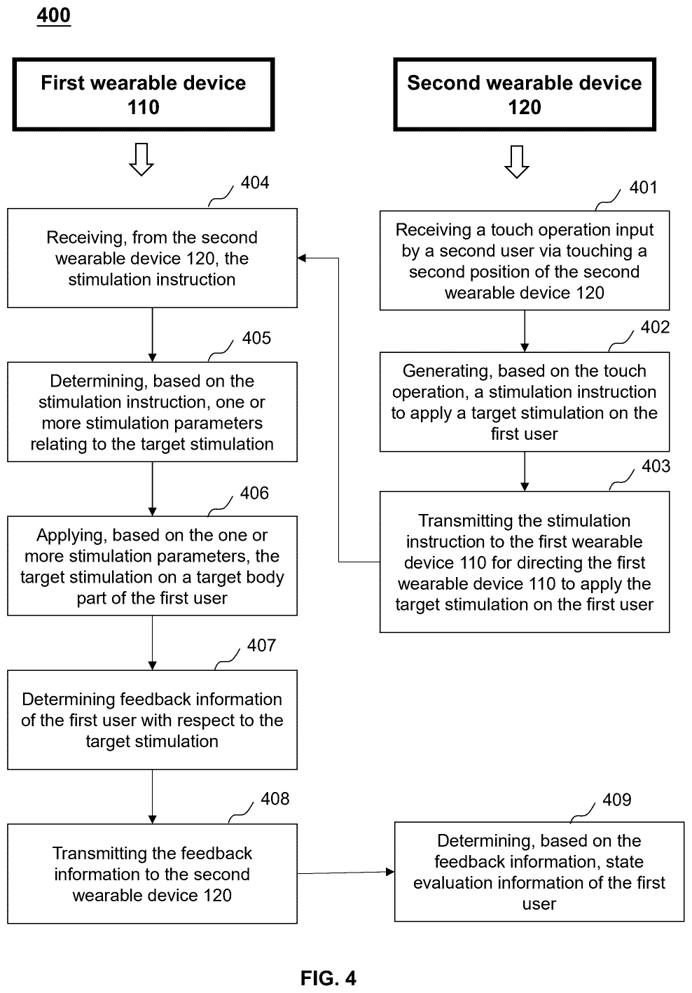

FIG. 4 is a flowchart illustrating an exemplary process 400 for remote diagnosis according to some embodiments of the present disclosure.

In 401, the second wearable device 120 may receive a touch operation (also referred to as a first touch operation) input by a second user via touching a second position of the second wearable device 120. In some embodiments, the operation 401 may be performed via the sensor 250 of the second wearable device 120. That is, the touch operation may be detected by the sensor 250 (e.g., a pressure sensor) of the second wearable device 120.

The second user may be a user (e.g., a doctor) who performs the remote diagnosis for a first user (e.g., a patient). The first user may be a user who needs to receive the remote diagnosis. The second wearable device 120 may be worn on a user (e.g., the second user or another user) or a human model. As user herein, the human model refers to a physical model (i.e., a dummy or a mannequin) made to imitate the human body (or a portion thereof). The human model may include a human model for a child, a human model for an adult, a human model for a male/female, a human model for a female, or the like. In some embodiments, the second user may select the human model suitable for the first user. By using the human body model, the second user does not need to wear the second wearable device 120, which may save time for the remote diagnosis and reduce the burden on the second user, thereby improving the efficiency of the remote diagnosis and the comfort of the second user.

For illustration purposes, the following descriptions describe the remote diagnosis process based on a second wearable device 120 worn the second user.

The second position of the second wearable device 120 may correspond to a target body part that needs to be examined for diagnosing the first user. For example, if the second user believes that the chest of the first user needs to be examined, the second user may touch a position of the second wearable device corresponding to the chest. As used herein, if a position of a wearable device covers a specific body part, the position of the wearable device may be deemed as being corresponding to the specific body part. In some occasions, a disease on a first body part may cause discomfort in a second body part. However, the discomfort only occurs in the second body part when the second body part is pressed, touched, or made corresponding movements. The second user may check both the first body part and the second body part according to clinical experience.

The second user may input the touch operation by touching the second position of the second wearable device 120. The term “touching” used herein refers to clicking, pressing, or any other action that is performed on a position of a wearable device. The second position may be also referred to as a touch position, which is a specific point or a specific region that is touched by the second user. The second wearable device 120 may determine information relating to the touch operation, such as information relating to the touch position, a touch strength, a touch angle, a touch duration, a body part that is touched, or the like, or any combination thereof. When the touch position is a specific point, a coordinate of the specific point may be determined. When the touch position is a specific region, a size of the specific region and position information of the specific region (e.g., a coordinate of the center of the specific region) may be determined. The touch strength may be an intensity of the force applied by the second user when touching the second position. The touch angle may refer to an angle (e.g., 30°, 35°, 90°, etc.) between a direction of the force exerted by the second user and a surface on which the force acts.

In 402, the second wearable device 120 may generate, based on the touch operation, a stimulation instruction to apply a target stimulation (also referred to as a first target stimulation) on the first user. In some embodiments, the operation 402 may be performed via the processor 210 of the second wearable device 120.

The stimulation instruction may be used to direct the first wearable device 110 to perform the target stimulation on the first user. The target stimulation is an action performed on the first user to stimulate the first user for examining or diagnosing the first user. The target stimulation can also be referred to as a target action or operation.

In some embodiments, the stimulation instruction may include one or more stimulation parameters (also referred to as one or more first stimulation parameters) relating to the target stimulation determined or obtained by the second wearable device 120. The one or more stimulation parameters may include a target body part, a stimulation strength, a stimulation angle, a stimulation frequency, a stimulation duration, a type, of the target stimulation, a first position of the first wearable device 110 corresponding to target body part, or the like, or any combination thereof.

The target body part refers to the body part to which the target stimulation is applied. The type of the target stimulation may include a pressing stimulation, an electrical stimulation, a temperature stimulation, a vibration stimulation, medicine application stimulation, or the like. The stimulation strength may be an intensity of the target stimulation applied by the first wearable device 110 to the first user. For examination, if the target stimulation is a pressing stimulation, the stimulation strength may be an intensity of the force applied by the first wearable device 110 to the first user.

In some embodiments, the second user may set at least a portion of the one or more stimulation parameters. For example, the second user may set the stimulation angle, the stimulation strength, the stimulation frequency, the stimulation duration, the type, etc. In some embodiments, the type of the target stimulation may be set by the second user. For example, the second wearable device 120 may include buttons corresponding to different types of stimulation, the second user may determine the type of the target stimulation via pressing one of the buttons.

In some embodiments, the second wearable device 120 may determine one or more stimulation parameters. For example, the second wearable device 120 may determine the touch strength of the touch operation of the second user on the second wearable device 120, and determine the stimulation strength based on the touch strength. Merely by way of example, the stimulation strength may be positively correlated to the touch strength of the second user on the second wearable device 120. In some embodiments, the stimulation strength may be equal to the touch strength of the second user on the second wearable device 120.

In some embodiments, the stimulation angle may be determined based on the touch angle. For example, the stimulation angle may be equal to the stimulation angle. In some embodiments, the stimulation duration may be determined based on the touch duration. For example, the stimulation duration may be equal to the touch duration. In some embodiments, the type of the target stimulation may be determined based on the target body part to be stimulated. For example, a corresponding relationship between body parts and types of stimulations may be previously determined, and the type of the target stimulation may be determined based on the corresponding relationship and the target body part. In some embodiments, the target body part may be determined based on the second position of the second wearable device 120 touched by the second user. Specifically, the second wearable device 120 may determine a body part of the user that wears the second wearable device 120 (e.g., the second user) or the human model corresponding to the second position (i.e., the body part of the second user covered by the second position), and designate the body part as the target body part. In some embodiments, the target body part may be determined based on the second position and a second corresponding relationship between positions of the second wearable device 120 and body parts of the user (or the human model). The second corresponding relationship may be determined in a similar manner as how a first corresponding relationship between positions of the first wearable device 110 and body parts of the first user is determined, which will be described in detail in connection with operation 405.

In some embodiments, the second wearable device 120 may determine the first position of the first wearable device 110 in a similar manner how the first wearable device 110 determines the first position of the first wearable device 110 as described in operation 405, and relevant descriptions can be found in operation 405.

In some embodiments, the stimulation instruction may include information relating to the touch operation, such as the touch position, the touch strength, the touch angle, etc. The information relating to the touch operation may be sent to the first wearable device 110 so that the first wearable device 110 can determine the stimulation parameter(s) based on the information.

In some embodiments, one or more stimulation parameters may be determined by the second wearable device 120, and one or more other stimulation parameters may be determined by the first wearable device 110.

In 403, the second wearable device 120 may transmit the stimulation instruction to the first wearable device 110 for directing the first wearable device 110 to apply the target stimulation on the first user.

In some embodiments, the operation 403 may be performed via the processor 210 and the communication port 240 of the second wearable device 120. For example, the processor 210 of the second wearable device 120 may direct the communication port 240 of the second wearable device 120 to transmit the stimulation instruction to the first wearable device 110 via the network 130.

In 404, the first wearable device 110 may receive, from the second wearable device, the stimulation instruction. In some embodiments, the operation 404 may be performed via the communication port 240 of the first wearable device 110.

The first wearable device 110 may be worn on the first user. As described in operation 401-402, the stimulation instruction may be invoked by the second user via touching the second position of the second wearable device 120.

In some embodiments, if the stimulation instruction includes information relating to the touch operation, such as the touch position, the touch strength, the touch angle, etc., the first wearable device 110 may perform operations 405 and 406 in sequence.

In some embodiments, if the stimulation instruction includes the one or more stimulation parameters relating to the target stimulation, the first wearable device 110 may directly perform operation 406.

In 405, the first wearable device 110 may determine, based on the stimulation instruction, one or more stimulation parameters relating to the target stimulation. In some embodiments, the operation 405 may be performed via the processor 210 of the first wearable device 110.

In some embodiments, as described operation 402, the one or more stimulation parameters may include a target body part, a stimulation strength, a stimulation angle, a stimulation frequency, a stimulation duration, a type, of the target stimulation, a first position of the first wearable device 110 corresponding to the target body part.

In some embodiments, the stimulation instruction includes information relating to the touch operation, such as information relating to the touch position (i.e., the second position), the touch strength, the touch angle, etc., and the first wearable device 110 may determine the one or more stimulation parameters based on the information relating to the touch operation in a similar manner as described in operation 402.

The first position of the first wearable device 110 may correspond to the target body part and the second position of the second wearable device 120. As used herein, if a position of the first wearable device 110 and a position of the second wearable device 120 correspond to the same body part, the position of the first wearable device 110 may be deemed as being corresponding to the position of the second wearable device 120. In some embodiments, the first position may be a position of a stimulation applying component of the first wearable device 110 that can perform the target stimulation and is located near the target body part of the first user.

In some embodiments, the stimulation instruction includes the target body part, the first position may be a position of the first wearable device 110 that covers the target body part. For example, if the target body part is the chest, the first position may be a position of the first wearable device 110 covers the chest of the first user.

In some embodiments, the first wearable device 110 may determine a first corresponding relationship between positions of the first wearable device 110 and body parts of the first user. Further, the first wearable device 110 may determine the first position of the first wearable device 110 based on the first corresponding relationship and the target body part. For example, if the target body part is the heart, the first wearable device 110 may determine a position of the first wearable device 110 corresponding to the heart of the first user as the first position of the first wearable device 110 according to the first corresponding relationship.

In some embodiments, the first wearable device 110 may obtain contour information of the first wearable device 110 after the first wearable device 110 is worn on the first user. For example, an image of the first wearable device 110 is collected, and the contour information of the first wearable device 110 is extracted from the image. As another example, position sensors of the first wearable device 110 may collect position information of different portions of the first wearable device 110 after the first wearable device 110 is worn on the first user, and the contour information may be determined based on the collected position information. Further, the first wearable device 110 may determine a surface model relating to the first user based on the contour information. Then, the first wearable device 110 may determine the first corresponding relationship based on the surface model. In some embodiments, the first wearable device 110 may identify body parts of the first user from the surface model. Exemplary body parts may include the head, the neck, the torso, the chest, the waist, buttocks, the left arm, the right arm, hands, the left lower limb, the right lower limb, feet, bone joints, or the like. Exemplary the bone joints may include shoulders (e.g., the left shoulder, the right shoulder), elbows (e.g., the left elbow, the right elbow), wrists (e.g., the left wrist, the right wrist), hands (e.g., the left hand, the right hand), the hip, knees (e.g., the left knee, the right knee), ankles (e.g., the left ankle, the right ankle), feet (e.g., the left foot, the right foot), or the like, or any combination thereof. The first wearable device 110 may determine a corresponding relationship between each body part and positions of the first wearable device 110 to obtain the first corresponding relationship.

In 406, the first wearable device 110 may perform, based on the one or more stimulation parameters, the target stimulation on the target body part of the first user. In some embodiments, the operation 406 may be performed via the stimulation applying component 260 of the first wearable device 110.

The target stimulation may be performed by one or more stimulation applying components 260 near the first position of the first wearable device 110. For example, a pressing stimulation is performed on the target body part of the first user via a pressing component. As another example, an electrical stimulation is performed on the target body part of the first user via an electrical stimulation component.

In some embodiments, the processor 210 of the first wearable device 110 may generate a stimulation signal based on the one or more stimulation parameters, and send the stimulation signal to one or more stimulation applying components 260 of the first wearable device 110 to apply the target stimulation on the target body part of the first user. The stimulation signal may include a pressure signal, an electrical stimulation signal, a temperature stimulation signal, a vibration signal, etc.

In 407, the first wearable device 110 may determine feedback information of the first user with respect to the target stimulation. In some embodiments, the operation 407 may be performed via the processor 210 of the first wearable device 110.

In some occasions, after the target stimulation is performed on the first user, the first user will have various physiological response (including feelings or reactions), such as pain, nausea, dizziness, an accelerated heartbeat, etc. For example, if the target stimulation is a pressing stimulation, the physiological response of the first user may include pain, tightness, swelling, etc. If the target stimulation is an electrical stimulation, the physiological response of the first user may include numb, slight tingling and itching, etc. If the target stimulation is a temperature stimulation, the physiological response of the first user may include may include hot, cold, etc. If the target stimulation is a vibration stimulation, the physiological response of the first user may include a strong vibration feeling, a weak vibration feeling, etc.

In some occasions, before the target stimulation is performed on the first user, the target body part of the first user may have some physiological response, after the target stimulation is performed on the first user, physiological response in the target body part may change (e.g., increase or relieve).

In some embodiments, the feedback information may include expression information, motion state information, physiological parameter information, voice information, image information, a discomfort strength (also referred to as discomfort degree), text information, etc., of the first user. The expression information may relate to facial expression of the first user before and after the target stimulation is performed on the target body part (e.g., after a starting time point of the target stimulation or after an ending time point of the target stimulation). The motion state information may include information relating to motions before and after the target stimulation is performed on the target body part. The physiological parameter information may include values of physiological parameters of the first user before and after the target stimulation is performed on the target body part. The image information may include images of the first user before and after the target stimulation is performed on the target body part.

In some embodiments, the first user may input the feedback information according to his/her physiological response, and the first wearable device 110 may obtain the feedback information feedbacked by the first user. For example, the first user may input voice feedback or text feedback to indicate that there is no pain in the target body part or the pain in the target body part increases.

In some embodiments, the first wearable device 110 may obtain one or more images of the first user captured after the target stimulation is performed on the target body part of the first user, and determine the feedback information based on the images. The image(s) of the first user may be collected via an image acquisition device mounted in a room where the first user is located. The image acquisition device may be and/or include any suitable device capable of capturing optical images of subjects located in a field of view of the image acquisition device. For example, the image acquisition device may include a camera (e.g., a digital camera, an analog camera, a binocular camera, etc.), a red-green-blue (RGB) sensor, an RGB-depth (RGB-D) sensor, a time-of-flight (TOF) camera, a depth camera, a structure light camera, a laser radar, or the like, or any combination thereof. For example, the first wearable device 110 may determine the expression information, the motion state information, etc., of the first user according to the image(s) of the first user as the feedback information. In some embodiments, the image(s) may be directly determined as the feedback information. In some embodiments, the first wearable device 110 may also obtain one or more images captured before the target stimulation is performed, and determine the feedback information based on the image(s) captured before and after the target stimulation is performed. For example, the change of the facial expression of the first subject may be determined as the feedback information.

In some embodiments, after the target stimulation is performed on the target body part of the first user, one or more physiological parameters of the first user may be measured using one or more physiological sensors of the first wearable device 110. The first wearable device 110 may determine the feedback information based on the one or more physiological parameters. The one or more physiological parameters may include a temperature, a blood pressure, a blood oxygen, a deformation (e.g., muscle contraction) of the target body part, a hardness of the target body part, an elasticity of the target body part, etc. For example, if the target stimulation is an electrical stimulation, the target body part may be deformed because the muscle of the target body part may contract. As another example, if the target stimulation is a pressure operation, the target body part may be deformed because the muscle of the target body part may concave inward.

In some embodiments, the first wearable device 110 may determine the discomfort degree (e.g., the pain degree) based on the physiological response of the first user after the target stimulation is performed on the target body part. For example, the first wearable device 110 may obtain a third corresponding relationship between the discomfort degree (e.g., pain) and the physiological response, and determine the discomfort degree according to the third corresponding relationship and the physiological response of the first user after the target stimulation is performed on the target body part. In some embodiments, the physiological response may be determined based on the expression of the first user, the deformation (e.g., muscle contraction) of the target body part, the physiological parameters of the first user, etc. The discomfort of the first user can be quantified by determining the discomfort degree of the target stimulation, so that the second user can learn the actual reaction of the first user more accurately, thereby improving the diagnosis and treatment accuracy.

In some embodiments, the third corresponding relationship may be generated before the target stimulation is performed on the target body part. Specifically, the discomfort may be divided into different discomfort degrees. For example, the pain may be divided into 1-9 discomfort degrees. As another example, the numbness may be divided into three discomfort degrees including a mild numbness, a moderate numbness, and a severe numbness. Before the target stimulation is performed on the target body part, the first wearable device 110 may perform a reference stimulation corresponding to each discomfort degree on the first user. The reference stimulation and the target stimulation have the same type. For example, both the reference stimulation and the target stimulation are electrical stimulations. When a reference stimulation corresponding to each discomfort degree is performed on the first user, the physiological response of the first user may be collected to generate a corresponding relationship between the physiological response of the first user and the discomfort degree. In addition, after a reference stimulation corresponding to each discomfort degree is performed on the first user, the first user may be informed with the discomfort degree so that the first user can learn about the feelings corresponding to different discomfort degrees. In this way, the first user can give more accurate feedback regarding his/her discomfort degree after the target stimulation is applied on him/her.

In some occasions, the first user may feedback her/his feelings relating to the target body part by touching the first wearable device 110. In some other occasions, after the target stimulation is performed on the target body part of the first user, physiological response occurs on another body part of the first user different from the target body part or the first user wants the second user to examine another body part, the first user may feedback information relating to the other body part by touching the first wearable device 110. In these cases, the feedback information may include a second touch operation input by the first user via touching a first reference position of the first wearable device 110. If the first user wants feedback her/his feelings relating to the target body part, the first reference position may be the same as or close to the first position of the first wearable device 110. If the first user may feedback the information relating to the other body part, the first reference position is different from the first position of the first wearable device 110.

The first wearable device 110 may generate a second stimulation instruction to apply a second target stimulation on the second user based on the second touch operation, and transmit the second stimulation instruction to the second wearable device 120. In some embodiments, the second stimulation instruction may be used for directing the second wearable device 120 to perform the second target stimulation on the second user. In some embodiments, similar to the first touch operation, the second touch operation may include a second touch position, a second touch strength, a second touch angle, a second touch duration, or the like, or any combination thereof. In some embodiments, the second stimulation instruction may include information relating to the second touch operation, such as the second touch position, the second touch strength, the second touch angle, etc.

In some embodiments, the second stimulation instruction may include one or more second stimulation parameters relating to the second target stimulation. The one or more second stimulation parameters may include a second target body part, a second stimulation strength, a second stimulation angle, a second stimulation frequency, a second stimulation duration, a second type, of the second target stimulation, a second reference position of the second wearable device 120 corresponding to the second target body part, or the like, or any combination thereof. In some embodiments, the determination of the one or more second stimulation parameters may be performed in a similar manner as that of the one or more first stimulation parameters described in operation 402.

In some embodiments, the second stimulation instruction may include the second stimulation strength that is determined based on the second touch strength. For example, the second stimulation strength is positively correlated to the second touch strength. It is understandable that the greater the second stimulation strength is, the greater the discomfort of the second user may be, and may even cause harm to the second user. Therefore, in some embodiments, before the second stimulation instruction is transmitted, the first wearable device 110 may determine whether the second stimulation strength of the second target stimulation is greater than a strength threshold. The strength threshold may be set manually by a user (e.g., the second user) according to an experience value or a default setting of the remote medical system 100. In response to determining that the second stimulation strength is greater than the strength threshold, the first wearable device 110 may adjust the second stimulation strength based on an adjustment coefficient and transmit the second stimulation instruction that includes the adjusted second stimulation strength. The adjusted second stimulation strength may be a product of the second stimulation strength and the adjustment coefficient. In some embodiments, the adjustment coefficient may be a constant such as 50%, 60%, 70%, etc. In some embodiments, the adjustment coefficient may be variable. The greater second stimulation strength is, the adjustment coefficient may be smaller. In this way, the discomfort caused by the second target stimulation on the second user may be reduced if the second stimulation strength is too great, thereby protecting the second user. In response to determining that the second stimulation strength is not greater than the strength threshold, the first wearable device 110 may transmit the second stimulation instruction that includes the second stimulation strength.

The second wearable device 120 may receive the second stimulation instruction from the first wearable device 110, and apply the second target stimulation on the second user in response to the second stimulation instruction. In some embodiments, the second target stimulation includes the one or more second stimulation parameters. In some embodiments, the second target stimulation includes information relating to the second touch operation, such as the second touch position, the second touch strength, the second touch angle, etc. The second wearable device 120 may determine the one or more second stimulation parameters in a similar manner as how the stimulation parameters are determined described in operation 405. Further, the second wearable device 120 may apply the second target stimulation on the second user based on the one or more second stimulation parameters. In some embodiments, the second wearable device 120 may apply the second target stimulation on the second user in a similar manner as how the target stimulation is performed on the first user as described in operation 406.

In some embodiments, the second stimulation instruction includes the adjusted second stimulation strength, the second wearable device 120 may apply the second target stimulation on the second user according to the adjusted second stimulation strength. In some embodiments, the second stimulation instruction includes the second stimulation strength, the second wearable device 120 may apply the second target stimulation on the second user according to the second stimulation strength.

In 408, the first wearable device 110 may transmit the feedback information to the second wearable device 120.

In some embodiments, the operation 408 may be performed via the processor 210 and the communication port 240 of the first wearable device 110. For example, the processor 210 of the first wearable device 110 may direct the communication port 240 of the first wearable device 110 to transmit the feedback information to the second wearable device 120 via the network 130.

Additionally or alternatively, the first wearable device 110 may transmit the feedback information to a display device of the second user. The display device may display the feedback information.

In 409, the second wearable device 120 may determine, based on the feedback information, state evaluation information of the target body part of the first user. In some embodiments, the operation 409 may be performed via the processor 210 of the second wearable device 120.

In some embodiments, the second wearable device 120 may obtain feedback information of the first user with respect to the target stimulation from the first wearable device 110, and determine state evaluation information of the target body part of the first user based on the feedback information.

The state evaluation information of the target body part may include whether the target body part has an abnormal reaction, a degree of abnormality of the target body part, or the like. In some embodiments, the second wearable device 120 may determine the expression change of the first user according to the expression information, and further determine whether the target body part has an abnormal reaction according to the expression change. For example, if the expression of the first user is change from expressionless to frowning and trembling according to the expression information, the second wearable device 120 may determine that the target body part has an abnormal reaction. In some embodiments, the second wearable device 120 may determine the motion change (e.g., from stillness to shaking) of the first user according to the motion state information. For example, if the first user changes from stillness to shaking according to the motion state information, the second wearable device 120 may determine that the target body part has an abnormal reaction. In some embodiments, the second wearable device 120 may determine whether the target body part has an abnormal reaction according to other information such as the voice information, the text information, etc., of the first user.

In some embodiments, the second wearable device 120 may determine the degree of abnormality of the target body part according to the discomfort degree of the first user. The higher the discomfort degree of the first user is, the degree of abnormality of the target body part may be greater.

In some embodiments, the second wearable device 120 may send the state evaluation information of the target body part to the display device of the second user for display.

In some embodiments, operations 408 and 409 may be omitted. The first wearable device 110 may determine state evaluation information of the target body part of the first user based on the feedback information, and send the state evaluation information to the display device of the second user. The state evaluation information may be displayed on the display device.

In some embodiments, the feedback information, the information relating to the target stimulation (e.g., the stimulation strength, a range of the stimulation strength), etc., may be displayed on the display device of the second user for diagnosis.

For example, after the target stimulation is performed, the stimulation signal can be converted into reference information that can be quantitatively described. Specifically, in some embodiments, the reference information may include specific parameters that can be displayed (e.g., physiological quantitative parameters). The physiological quantitative parameters may be used to characterize a discomfort degree, a health degree, etc. of the first user. More specifically, the physiological quantitative parameters may include a stimulation signal interval corresponding to the stimulation signal, or the discomfort degree corresponding to the stimulation signal. When the reference information includes the physiological quantitative parameters, the reference information may be perceived by the second user in the following manner. The physiological quantitative parameters are displayed on a human-computer interaction interface via the second wearable device 120. The human-computer interaction interface may include a command interface, a menu interface, a graphical user interface, etc. In some embodiments, the human-computer interaction interface may include multiple display components, and the display components may be used to display the physiological quantitative parameters. For example, the pain degree may be displayed as 3 level or a pain index of 0.5. Of course, the display components may also display an image of the target body part of the first user, and may mark the discomfort degree on the image. In this way, the second user can clearly know the quantitative characteristics of the target body part of the first user by observing the display data in the human-computer interaction interface, and intuitively analyze the condition of the first user and conduct online diagnosis and treatment. In some embodiments, multiple candidate stimulation signal intervals may be preset, and endpoints of each candidate stimulation signal interval are different. Then, the stimulation signal interval corresponding to the stimulation signal can be determined from the multiple candidate stimulation signal intervals. For example, the candidate stimulation signal intervals may include an interval of 0-0.2 mA, an interval of 0.2-0.4 mA, and an interval of 0.4-0.6 mA. When the stimulation signal is 0.3 mA, the stimulation signal interval may be determined to be the interval of 0.2-0.4 mA.

In some embodiments, the second user may determine a diagnosis result of the first user based on information relating to the first user such as the state evaluation information of the target body part, the feedback information, the information relating to the target stimulation, etc. In some embodiments, according to actual needs, the process 400 may be performed on each of one or more other target body parts to determine state evaluation information of the one or more other target body parts. The information relating to the first user may further include the state evaluation information of the one or more other target body parts.

In some embodiments, if the second wearable device 120 performs the second target stimulation on the second user based on the second stimulation instruction, the information relating to the first user may further include feelings of the second user regarding the second target stimulation.

In some embodiments, the diagnosis result may include a position of a lesion of the first user, a type of the lesion, a severity of the lesion, or the like. In some embodiments, the second user may obtain an image of the first user, and annotate the lesion of the first user on the image according to the position of the lesion in the diagnosis result. Alternatively or optionally, the image of the first user may be obtained and the lesion of the first user may be annotated on the image via the second wearable device 120. The image annotated with the lesion may be displayed on the display device of the second user. In some embodiments, the lesion and other regions of the first user may be displayed using different color. In some embodiments, the lesion may be displayed according to the severity of the lesion. For example, if the severity of the lesion is relatively high, the color of the lesion may be red; if the severity of the lesion is medium, the color of the lesion may be yellow; if the severity of the lesion is relatively low, the color of the lesion may be green. In some embodiments, the image of the first user may be obtained via a digital twin technology. The image of the first user may include a virtual digital model of the first user. The virtual digital model may be constructed based on status data, behavioral data, physiological measurement data, and other data of the first user. The virtual digital model can truly reflect the behavior, status, etc., of the first user in the real environment in real time.

In some embodiments, in order to simulate all aspects of outpatient service as much as possible, the 3D videos of the first user and/or the second user may also be displayed to realize the visual stimulation function. Specifically, during the remote diagnosis, a three-dimensional (3D) video of the first user may be displayed on the display device of the second user. The body characteristic information and audio information of the first user may be collected through a video recording device. The video recording device may include a 3D video recording device such as a panoramic camera, a 360° camera, a 360° VR panoramic camera, a 720° VR panoramic camera, etc. The body characteristic information may include pose information, behavior information, etc. The pose information may include position information and posture information of the first user. The posture information may include a standing posture, a sitting posture, a lying posture, a golden rooster independent posture, etc. The behavior information may include behaviors of the first user such as standing, running, pacing, etc. In other embodiments, the video recording device may also include a 2D video recording device, such as a high-definition camera. When the video recording device is a 2D video recording device, the 2D videos generated by the 2D video recording device may be spliced into a 3D video. The 3D video may capture the first user in all directions. The 3D video may be displayed in a virtual environment. The 3D video may include, for example, a virtual reality (VR) signal, an augmented reality (AR) signal, a mixed reality (MR) signal, etc. In some embodiments, the video recording device may be installed at a designated position above the first user. In other embodiments, the video recording device may be installed at any position in the environment where the first user is located that can capture the first user in all directions. This application is not limited here. In some embodiments, in order to allow the second user to fully observe the first user, the 3D video may be displayed in a virtual environment via a device for projecting the 3D video.

In some embodiments, during the remote diagnosis, a three-dimensional (3D) video of the second user may be displayed on a display device of the first user.

As described elsewhere in the present disclosure, the conventional remote medical systems cannot completely simulate all aspects of outpatient service. Compared with the conventional remote medical systems, the remote medical systems and methods of the present disclosure determine the state evaluation information of one or more target body parts of the patient through the first wearable device 110 and the second wearable device 120, and further determine the diagnosis result based on the state evaluation information of one or more target body parts of the patient, which can completely simulate the palpation process, thereby improving the diagnosis and treatment accuracy. In addition, in some embodiments, various types of feedback information (e.g., expression information, motion state information, physiological parameter information, voice information, image information, a strength of the discomfort, text information) can be collected by the first wearable device 110 and sent to the second wearable device 120 so that the second user can learn the actual reaction of the first user more accurately, thereby improving the diagnosis and treatment accuracy.

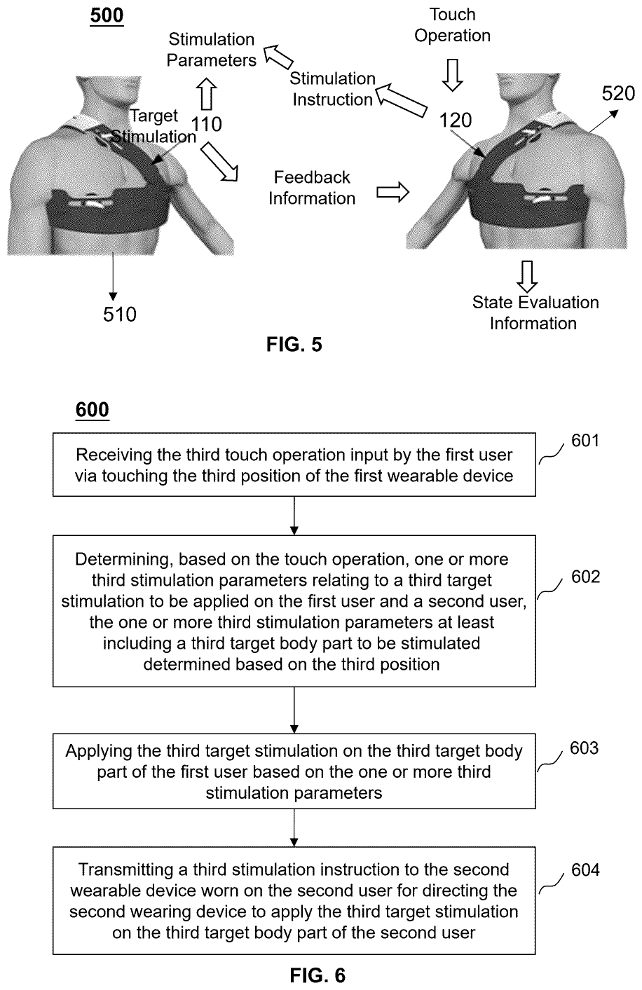

FIG. 5 is a schematic diagram illustrating an exemplary process 500 for remote diagnosis according to some embodiments of the present disclosure.

As shown in FIG. 5, the first wearable device 110 is worn on a first user 510 to be examined, and the second wearable device 120 is worn on a second user 520 who performs remote diagnosis for the first user 510. The second wearable device 120 may receive a touch operation input by the second user 520 via touching a position of the second wearable device 120. Further, the second wearable device 120 may generate a stimulation instruction to apply a target stimulation (e.g., a press) on the first user 510 based on the touch operation and transmit the stimulation instruction to the first wearable device 110. After the first wearable device 110 receives the stimulation instruction, the first wearable device 110 may determine one or more stimulation parameters relating to the target stimulation based on the stimulation instruction. Then, the first wearable device 110 may apply the target stimulation on a target body part of the first user 510 based on the one or more stimulation parameters. The first wearable device 110 may determine feedback information of the first user 510 with respect to the target stimulation, and send the feedback information to the second wearable device 120. The second wearable device 120 may determine the state evaluation information of the target body part of the first user based on the feedback information.

In some embodiments, the feedback information may include a second touch operation input by the first user via pressing the first wearable device 110. The first wearable device 110 may generate a second stimulation instruction to apply a second target stimulation on the second user 520 based on the second touch operation, and transmit the second stimulation instruction to the second wearable device 120. After the second wearable device 120 receives the second stimulation instruction, the second wearable device 120 may apply the second target stimulation on the second user 520 based on the second stimulation instruction. In such cases, the second user 520 can feel the discomfort of the first user 510 and accurately determine uncomfortable body part of the first user 510 and/or the discomfort degree of the first user 510.

FIG. 6 is a flowchart illustrating an exemplary process 600 for remote diagnosis according to some embodiments of the present disclosure.

In some occasions, a first user needs to point out an uncomfortable body part (i.e., a target body part) (e.g., the heart, the brain) of the first user, or inform a second user of a discomfort degree of the first user, etc. The first user may perform a third touch operation via touching a third position of the first wearable device 110.

In 601, the first wearable device 110 may receive the third touch operation input by the first user via touching the third position of the first wearable device 110. In some embodiments, the operation 601 may be performed via the sensor 250 of the first wearable device 110. That is, the touch operation may be detected by the sensor 250 (e.g., a pressure sensor) of the first wearable device 110.

The first user may include any subject that needs to receive the remote diagnosis. The first wearable device 110 may be worn on the first user.

In some embodiments, similar to the first touch operation, information relating to the third touch operation may be determined, such as a third touch position, a third touch strength, a third touch angle, a third touch duration, or the like, or any combination thereof. For example, the first user may act on the first wearable device 110 with a force by touching, clicking, etc., the third position of the first wearable device 110. In some embodiments, the discomfort degree and the third touch strength be positively correlated. That is, the greater the discomfort degree is, the greater the third touch strength may be. In some embodiments, the discomfort degree and the third touch duration be positively correlated. That is, the greater the discomfort degree is, the greater the third touch duration may be.

In 602, the first wearable device 110 may determine, based on the third touch operation, one or more third stimulation parameters relating to a third target stimulation to be performed on the first user and a second user, the one or more third stimulation parameters at least including a third target body part determined based on the third position. In some embodiments, the operation 602 may be performed via the processor 210 of the first wearable device 110.

The third target body part refers to the body part (e.g., the chest) to which the third target stimulation is applied. The third target body part may be the body part corresponding to the third position of the first wearable device 110, i.e., the body part covered by the third position of the first wearable device 110.

In some embodiments, the first wearable device 110 may generate a third stimulation instruction based on the third touch operation. The third stimulation instruction may be used for directing the first wearable device 110 to perform the third target stimulation on the first user and directing the second wearable device 120 to perform the third target stimulation on the second user.

In some embodiments, the third stimulation instruction may include the one or more third stimulation parameters relating to the third target stimulation. The one or more third stimulation parameters may include the third target body part, a third stimulation strength, a third stimulation angle, a third stimulation frequency, a third stimulation duration, a third type, etc., of the third target stimulation, the third position of the first wearable device 110, a fourth position of the second wearable device 110 corresponding to the third target body part, or the like, or any combination thereof. In some embodiments, the determination of the one or more third stimulation parameters may be performed in a similar manner as that of the one or more first stimulation parameters described in operation 402 and operation 405.

In 603, the first wearable device 110 may perform the third target stimulation on the third target body part of the first user based on the one or more third stimulation parameters. In some embodiments, the operation 603 may be performed via the stimulation applying component 260 of the first wearable device 110.

In some embodiments, the operation 603 may be performed in a similar manner as operation 406.

In some embodiments, after the third target stimulation is performed on the third target body part of the first user, the first wearable device 110 may determine feedback information of the first user with respect to the third target stimulation in a similar manner as how the feedback information is determined as described in operation 407. For example, the first wearable device 110 may obtain an image of the first user after the third target stimulation is performed on the third target body part of the first user, and further determine the feedback information based on the image. As another example, the first wearable device 110 may measure one or more physiological parameters of the first user after the target stimulation is performed on the third target body part of the first user using one or more physiological sensors mounted on the first wearable device 110, and further determine the feedback information based on the one or more physiological parameters.

In some embodiments, the first wearable device 110 may determine state evaluation information of the third target body part of the first user based on the feedback information, and send the state evaluation information to a display device of the second user or the second wearable device 120. In some embodiments, the first wearable device 110 may send the feedback information to the display device of the second user or the second wearable device 120. The second wearable device 120 or the second user may determine the state evaluation information of the third target body part of the first user based on the feedback information. The determination of the state evaluation information of the third target body part of the first user may be performed in a similar manner as how the state evaluation information of the target body part is determined as described in operation 409.

In 604, the first wearable device 110 may transmit the third stimulation instruction to the second wearable device 120 worn on the second user for directing the second wearable device 120 to perform the third target stimulation on the third target body part of the second user.

In some embodiments, the operation 604 may be performed via the processor 210 and the communication port 240 of the first wearable device 110. For example, the processor 210 of the first wearable device 110 may direct the communication port 240 of the first wearable device 110 to transmit the stimulation instruction to the second wearable device 120 via the network 130.

In some embodiments, after the second wearable device 120 receives the third stimulation instruction, the second wearable device 120 may perform the third target stimulation on the third target body part of the second user in a similar manner how the target stimulation is performed on the first user as described in operation 406.

In some embodiments, the third stimulation instruction may include the third stimulation strength that is determined based on the third touch strength. For example, the third stimulation strength is positively correlated to the third touch strength. It is understandable that the greater the third stimulation strength is, the greater the discomfort of the second user may be, and may even cause harm to the second user. Therefore, in some embodiments, before the third stimulation instruction is transmitted, the first wearable device 110 may determine whether the third stimulation strength of the third target stimulation is greater than a strength threshold. The strength threshold may be set manually by a user (e.g., the second user) according to an experience value or a default setting of the remote medical system 100. In response to determining that the third stimulation strength is greater than the strength threshold, the first wearable device 110 may adjust the third stimulation strength based on an adjustment coefficient and transmit the third stimulation instruction that includes the adjusted third stimulation strength. The adjusted third stimulation strength may be a product of the third stimulation strength and the adjustment coefficient. In some embodiments, the adjustment coefficient may be a constant such as 50%, 60%, 70%, etc. In some embodiments, the adjustment coefficient may be variable. The greater third stimulation strength is, the adjustment coefficient may be smaller. The second wearable device 120 may perform the third target stimulation on the third target body part of the second user according to the adjusted third stimulation strength. In response to determining that the third stimulation strength is not greater than the strength threshold, the first wearable device 110 may transmit the third stimulation instruction that includes the third stimulation strength. The second wearable device 120 may perform the third target stimulation on the third target body part of the second user according to the third stimulation strength. In this way, the discomfort caused by the third target stimulation may be reduced, thereby protecting the second user.

In some embodiments, the second user may determine a diagnosis result of the first user based on the state evaluation information of the third target body part and feelings of the second user for the third target stimulation. In some embodiments, according to actual needs, the process 600 may be performed on one or more other target body parts of the first user to determine state evaluation information of the one or more other target body parts. The second user may determine the diagnosis result of the first user based on the state evaluation information of the third target body part and the one or more other target body parts of the first user, and feelings of the second user for the third target stimulation and other target stimulations corresponding to the one or more other target body parts.

In some embodiments, the diagnosis result may include a position of a lesion of the first user, a type of the lesion, a severity of the lesion, or the like. In some embodiments, an image of the first user may be obtained, and the lesion of the first user may be annotated on the image according to the position of the lesion in the diagnosis result.

According to process 600, the same target stimulation (i.e., the third target stimulation) may be performed on the same body part of the first user and the second user, so that the second user also can accurately determine uncomfortable body part of the first user and the discomfort degree of the first user even if the first user and the second user are in different spaces, which may allow the second user to empathize with the first user, thereby greatly improving the accuracy and the efficiency of the remote diagnosis.

It should be noted that the processes 400 and 600 and the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure. For example, the operations of the illustrated processes 400 and 600 are intended to be illustrative. In some embodiments, the processes 400 and 600 may be accomplished with one or more additional operations not described, and/or without one or more of the operations discussed. Additionally, the order in which the operations of the processes 400 and 600 and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, 15%, 110%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

1. A method, implemented on a first wearable device worn on a first user to be examined, comprising:
receiving, from a second wearable device, a stimulation instruction to apply a target stimulation on the first user, the stimulation instruction being invoked by a second user via touching a second position of the second wearable device;
determining, based on the stimulation instruction, one or more stimulation parameters relating to the target stimulation, the one or more stimulation parameters at least including a target body part to be stimulated determined based on the second position;
performing, based on the one or more stimulation parameters, the target stimulation on the target body part of the first user; and
determining feedback information of the first user with respect to the target stimulation.

2. The method of claim 1, wherein the performing, based on the one or more stimulation parameters, the target stimulation on the target body part of the first user comprises:
determining a corresponding relationship between positions of the first wearable device and body parts of the first user;
determining a first position of the first wearable device corresponding to the target body part based on the corresponding relationship and the target body part; and
performing the target stimulation on the target body part of the first user based on the first position and the one or more stimulation parameters.

3. The method of claim 2, wherein the determining a corresponding relationship between positions of the first wearable device and body parts of the first user comprises:
obtaining contour information of the first wearable device;
determining a surface model relating to the first user based on the contour information; and
determining the first corresponding relationship based on the surface model.

4. The method of claim 1, wherein the stimulation instruction includes touch strength of the touch of the second user on the second position, and the one or more stimulation parameters further include a stimulation strength of the target stimulation determined based on the touch strength.

5. The method of claim 1, wherein the second wearable device is worn on the second user, the feedback information includes a touch operation input by the first user via touching the first wearable device, and the method further comprises:
generating, based on the touch operation, a second stimulation instruction to apply a second target stimulation on the second user;
transmitting the second stimulation instruction to apply the second target stimulation on the second user to the second wearable device.

6. The method of claim 5, wherein the transmitting the second stimulation instruction to apply the second target stimulation on the second user to the second wearable device comprises:
determining whether a second stimulation strength of the second target stimulation is greater than a strength threshold; and
in response to determining that the second stimulation strength is greater than the strength threshold, adjusting the second stimulation strength based on an adjustment coefficient and transmitting the second stimulation instruction that includes the adjusted second stimulation strength; or in response to determining that the second stimulation strength is not greater than the strength threshold, transmitting the second stimulation instruction that includes the second stimulation strength.

7. The method of claim 1, wherein the determining feedback information of the first user with respect to the target stimulation comprises:
obtaining one or more images of the first user captured after the target stimulation is performed on the target body part of the first user; and
determining the feedback information based on the image.

8. The method of claim 1, wherein the determining feedback information of the first user with respect to the target stimulation comprises:
measuring, using one or more physiological sensors mounted on the first wearable device, one or more physiological parameters of the first user after the target stimulation is performed on the target body part of the first user; and
determining the feedback information based on the one or more physiological parameters.

9. The method of claim 1, further comprising:
determining state evaluation information of the first user based on the feedback information, and sending the state evaluation information to a display device of the second user; or
sending the feedback information to the display device of the second user or the second wearable device.

10. The method of claim 1, wherein the second wearable device is worn on the second user or a human model.

11. A method, implemented on a second wearable device, comprising:
receiving a touch operation input by a second user via touching a second position of the second wearable device;
generating, based on the touch operation, a stimulation instruction to apply a target stimulation on a first user; and
transmitting the stimulation instruction to a first wearable device for directing the first wearable device to apply the target stimulation on the first user.

12. The method of claim 11, further comprising:
determining a touch strength of the touch operation of the second user on the second wearable device.

13. The method of claim 12, wherein the stimulation instruction further comprises a stimulation strength of the target stimulation determined based on the touch strength.

14. The method of claim 11, further comprising:
obtaining, from the first wearable device, feedback information of the first user with respect to the target stimulation; and
determining, based on the feedback information, state evaluation information of the first user.

15-16. (canceled)

17. A method, implemented on a first wearable device worn on a first user to be examined, comprising:
receiving a touch operation input by the first user via touching a first position of the first wearable device;
determining, based on the touch operation, one or more stimulation parameters relating to a target stimulation to be performed on the first user and a second user, the one or more stimulation parameters at least including a target body part to be stimulated determined based on the first position;
performing the target stimulation on the target body part of the first user based on the one or more stimulation parameters; and
transmitting a stimulation instruction to a second wearable device worn on the second user for directing the second wearable device to apply the target stimulation on the target body part of the second user.

18. The method of claim 17, wherein the one or more stimulation parameters further include a stimulation strength of the target stimulation, and the method further comprises:
determining whether the stimulation strength of the target stimulation is greater than a strength threshold; and
in response to determining that the stimulation strength is greater than the strength threshold, adjusting the stimulation strength based on an adjustment coefficient and transmitting the stimulation instruction that includes the adjusted strength; or in response to determining that the stimulation strength is not greater than the strength threshold, transmitting the stimulation instruction that includes the stimulation strength.

19. The method of claim 17, further comprising:
determining feedback information of the first user with respect to the target stimulation.

20. The method of claim 19, wherein the determining feedback information of the first user with respect to the target stimulation comprises:
obtaining one or more images of the first user after the target stimulation is performed on the target body part of the first user; and
determining the feedback information based on the one or more images.

21. The method of claim 19, wherein the determining feedback information of the first user with respect to the target stimulation comprises:
measuring, using one or more physiological sensors mounted on the first wearable device, one or more physiological parameters of the first user after the target stimulation is performed on the target body part of the first user; and
determining the feedback information based on the one or more physiological parameters.

22. The method of claim 19, further comprising:
determining state evaluation information of the target body part of the first user based on the feedback information, and sending the state evaluation information to a display device of the second user; or
sending the feedback information to the display device of the second user or the second wearable device.

23-29. (canceled)
